(19) United States
(12) Patent Application Publication
(10) Pub. No.: US 2025/0326781 A1
(43) Pub. Date: Oct. 23, 2025

(54) METHOD FOR PRODUCING SUGAR

(71) Applicant: OSAKA UNIVERSITY, Suita-shi, Osaka (JP)

(72) Inventors: Shuji NAKANISHI, Suita-shi, Osaka (JP); Hiro TABATA, Suita-shi, Osaka (JP); Hiroaki NISHIJIMA, Suita-shi, Osaka (JP); Genta CHIKATANI, Suita-shi, Osaka (JP)

(21) Appl. No.: 18/700,648

(22) PCT Filed: Oct. 12, 2022
(86) PCT No.: PCT/JP2022/038093

§ 371 Date: Oct. 23, 2024

(30) Foreign Application Priority Data
Oct. 12, 2021 (JP) 2021-167725

(51) Int. Cl. C07H 1/00; B01J 23/28; B01J 23/30; C07B 61/00; C07H 3/02

(52) U.S. Cl. CPC C07H 1/00; B01J 23/28; B01J 23/30; C07B 61/00; C07H 3/02

(57) ABSTRACT

The purpose of the present invention is to provide a technology useful for sugar synthesis, the technology using formaldehyde as the main raw material and allowing for selective promotion of the formose reaction. This method for producing sugar comprises a step for allowing an action to occur under neutral conditions, using formaldehyde as a substrate, a sugar as an initiator, and as a catalyst, a compound that, under neutral conditions, is capable of deprotonating an hydroxyl group of erythrose and is not capable of deprotonating water.

### TECHNICAL FIELD

The present invention relates to a method for producing a sugar using a selective formose reaction under neutral conditions and a catalyst used therefor.

### BACKGROUND ART

While the world population is said to increase to 9.8 billion people by 2050, it is estimated that the production increase of grain feed remains about 1.5 times. It is, therefore, inevitable that animal proteins will be greatly insufficient in the future, and cell/tissue culture engineering and techniques for utilizing insects and the like have attracted great attention.

In breeding organisms or culturing cells, sugars are essential as primary energy, and the production of the sugars depends on agriculture (that is, photosynthesis) without exception. However, since current agriculture requires a large amount of agricultural water and agricultural land, an approach is desired in which the primary energy for protein production is not solely dependent on photosynthesis.

In view of the above problems, it is desired to establish an innovative feed production system using power derived from renewable energy as primary energy. Elemental techniques for establishing this system include (1) electrochemical CO₂/HCHO conversion, (2) chemical HCHO/sugar conversion, and (3) sugar/feed conversion by microbial processes.

In relation to the elemental technique (2), the formose reaction in which sugars are produced by heating an aqueous formaldehyde solution under basic conditions is known, and there is interest in it from the viewpoint of upgrading of organic substances, the origin of life, and the like. However, in the formose reaction, since as many as several tens of kinds of substances are non-selectively obtained by competition reactions and decomposition of products, industrial application is still difficult.

Allowing the formose reaction to proceed selectively is an important task in enhancing its usefulness, and various approaches have been attempted to address this task. For example, Non-Patent Document 1 reports that an aldol reaction between glycolaldehyde and formaldehyde proceeds by using hydroxyapatite as a catalyst, and sugar products containing ribose are obtained. In addition, Non-Patent Document 2 reports that erythrulose and 3-pentulose are selectively obtained by causing an aldol reaction between formaldehyde and dihydroxyacetone.

### PRIOR ART DOCUMENT

### Non-Patent Documents

Non-Patent Document 1: Org. Biomol. Chem., 2017, 15, 8888-8893
Non-Patent Document 2: Kinetics and Catalysis vol 48, 550-555 (2007)

### SUMMARY OF THE INVENTION

### Problems to be Solved by the Invention

A typical reaction route of the formose reaction is shown in FIG. 1. The reaction begins with the formation of glycolaldehyde (C2) by dimerization of formaldehyde, various sugar products are produced through a chain cross-aldol reaction. Regeneration of glycolaldehyde by cleavage of erythrose (C4) occurs, so that the reaction proceeds autocatalytically.

Although there are various theories as to whether the dimerization reaction of formaldehyde actually proceeds, the formose reaction proceeds under basic conditions by adding a small amount of glycolaldehyde as an initiator without other catalysts. That is, the reactions of k2 to k4 in FIG. 1 proceed by OH, and complicated products are obtained. Thus, it can be said that in the formose reaction using OH as a catalyst, a decrease in selectivity under basic conditions is inevitable. Then, if the formose reaction can proceed under neutral conditions, it can be expected that a decrease in selectivity caused by OH⁻ is suppressed.

Techniques proposed in Non-Patent Document 1 and Non-Patent Document 2 allow the formose reaction to proceed under neutral conditions, and it is considered that the techniques allow the reaction to proceed selectively to some extent. However, these techniques need to use high-cost substances such as glycolaldehyde and dihydroxyacetone as raw material substrates. Use of these high-cost substances as raw materials is problematic in that it goes against the gist of carbon upgrading.

Then, it is desired to increase the selectivity of the formose reaction using inexpensive formaldehyde as a main raw material. In addition, in view of the fact that formaldehyde is obtained by electrochemical reduction of CO₂, the formose reaction using formaldehyde as a main raw material is also desirable from the viewpoint of carbon neutrality.

It is therefore an object of the present invention to provide a technique useful for synthesis of a sugar that uses formaldehyde as a main raw material and allows the formose reaction to proceed further selectively.

### Means for Solving the Problem

The present inventor has focused on an approach to promoting the regeneration of intermediate products by the retro-aldol reaction under neutral conditions in order to allow a selective formose reaction to proceed using formaldehyde as a main raw material. As a result, the present inventor has found that when a predetermined catalyst promotes the retro-aldol reaction under neutral conditions and is further combined with the use of a sugar as an initiator, it is possible to allow the formose reaction to proceed selectively using formaldehyde as a main raw material, and its high usefulness in the synthesis of a sugar is achieved.

That is, the present invention provides inventions of the following aspects.
Item 1. A method for producing a sugar, the method including a step of allowing a compound having an ability to deprotonate a hydroxyl group of erythrose and not having an ability to deprotonate water to act as a catalyst under neutral conditions, using formaldehyde as a substrate and a sugar as an initiator.
Item 2. The method according to item 1, in which when the catalyst is prepared as an aqueous solution having a concentration of 60 mM, the catalyst has a base strength of pH 4 to 9 at 25° C.
Item 3. The method according to item 1 or 2, in which the catalyst is a compound containing a metal selected from the group consisting of a Group 5 element, a Group 6 element, and a Group 13 element, and/or an organic base.
Item 4. The method according to item 3, in which the compound containing the metal is an oxide of a Group 5 element, a Group 6 element, or a Group 13 element, an oxoacid of a Group 5 element, a Group 6 element, or a Group 13 element and/or a salt of the oxoacid, and/or a complex of a Group 5 element, a Group 6 element, or a Group 13 element.
Item 5. The method according to item 3 or 4, in which the metal is selected from the group consisting of niobium, molybdenum, tungsten, and aluminum.
Item 6. The method according to any one of items 3 to 5, in which the compound containing the metal is selected from the group consisting of molybdate, tungstate, molybdenum oxide, niobate, and aluminum oxide.
Item 7. The method according to any one of items 1 to 6, in which the catalyst has a form that is a form of the compound itself, a form in which the compound is supported on an insoluble carrier, and/or a form of an insoluble solid phase doped with an ion to be supplied to a reaction system by containing the compound as a constituent element of a material.
Item 8. The method according to any one of items 1 to 7, in which the initiator is used in a ratio of from 0.1 to 50 mmol to 1 mol of the substrate.
Item 9. The method according to any one of items 1 to 8, in which a substrate other than formaldehyde is not added.
Item 10. The method according to any one of items 1 to 9, further including a step of obtaining the formaldehyde by electrolysis of carbon dioxide,
in which the step of obtaining the formaldehyde and the step of allowing the compound to act as the catalyst are performed in a system of the electrolysis.
Item 11. A catalyst that contains a compound having an ability to deprotonate a hydroxyl group of erythrose and not having an ability to deprotonate water, and is used in production of a sugar under neutral conditions using formaldehyde as a substrate and a sugar as an initiator.
Item 12. A sugar production system including: a carbon dioxide electrolysis apparatus that forms formaldehyde from carbon dioxide; and the catalyst according to item 11.

### Advantages of the Invention

According to the present invention, it is possible to provide a technique useful for synthesis of a sugar that uses formaldehyde as a main raw material and can further selectively progress the formose reaction.

### BRIEF DESCRIPTION OF THE DRAWINGS

FIG. 1 shows the formose reaction pathway.

FIG. 2 shows temporal changes in the concentration of each of erythrose (C4) and a product (glycolaldehyde (C2)) in the case of a retro-aldol reaction using erythrose as the substrate performed using disodium tungstate as the catalyst.

FIG. 3 shows temporal changes in the amount of formaldehyde (HCHO) consumed and the concentration of glycolaldehyde (C2) produced in the case of the formose reaction performed using glycolaldehyde as the initiator and disodium tungstate as the catalyst.

FIG. 4 shows analysis results of the produced sugars at the time 99 mol % formaldehyde as the substrate was consumed in the case of the formose reaction performed using glycolaldehyde as the initiator and disodium tungstate as the catalyst.

FIG. 5 shows analysis results of the produced sugars in the case of the formose reaction performed using glycolaldehyde as the initiator and disodium molybdate as the catalyst.

FIG. 6 shows temporal changes in the amount of formaldehyde (HCHO) consumed and the concentration of glycolaldehyde (C2) produced in the case of the formose reaction performed using glycolaldehyde as the initiator and molybdenum oxide as the catalyst.

FIG. 7 shows analysis results of the produced sugars in the case of the formose reaction performed using glycolaldehyde as the initiator and aluminum oxide as the catalyst.

FIG. 8 shows analysis results of the produced sugars in the case of the formose reaction performed using glycolaldehyde as the initiator and barium tungstate solubilized with EDTA·2Na as the catalyst.

FIG. 9 shows analysis results of the produced sugars in the case of the formose reaction performed using glycolaldehyde as the initiator and potassium niobate as the catalyst.

FIG. 10 shows analysis results of the produced sugars in the case of the formose reaction performed using glycolaldehyde as the initiator and tungstate anion immobilized on an insoluble carrier as the catalyst.

FIG. 11 shows analysis results of the produced sugars at the time 99 mol % of formaldehyde as the substrate was consumed in the case of the formose reaction performed using dihydroxyacetone as the initiator and disodium tungstate as the catalyst.

FIG. 12 shows analysis results of the produced sugars at the time 99 mol % formaldehyde as the substrate was consumed in the case of the formose reaction performed using fructose as the initiator and disodium tungstate as the catalyst.

FIG. 13 shows analysis results of the produced sugars at the time 99 mol % of formaldehyde as the substrate was consumed in the case of the formose reaction performed using sorbose as the initiator and disodium tungstate as the catalyst.

FIG. 14 shows analysis results of the produced sugars at the time 99 mol % of formaldehyde as the substrate was consumed in the case of the formose reaction performed using glucose as the initiator and disodium tungstate as the catalyst.

FIG. 15 shows analysis results of the produced sugars at the time 99 mol % of formaldehyde as the substrate was consumed in the case of the formose reaction performed using sucrose as the initiator and disodium tungstate as the catalyst.

FIG. 16 shows analysis results of the produced sugars at the time 99 mol % of formaldehyde as the substrate was consumed in the case of the formose reaction performed using starch as the initiator and disodium tungstate as the catalyst.

### EMBODIMENTS OF THE INVENTION

### 1. Method for Producing Sugar

The method for producing a sugar of the present invention includes the step of allowing a predetermined catalyst to act under neutral conditions using formaldehyde as a substrate and a sugar as an initiator (hereinafter, also referred to as “formose reaction step”). The method for producing a sugar of the present invention constructs the formose reaction system exemplified in FIG. 1 to produce a sugar.

In the formose reaction step, a reaction mixture containing formaldehyde, a sugar, and a predetermined catalyst is subjected to reaction conditions for allowing the formose reaction to proceed, thereby generating a sugar in a mixture of reactants and reaction products.

### 1-1. Substrate

The amount of formaldehyde as the substrate added in the reaction mixture is not particularly limited, but is, for example, 0.01 to 5 M, preferably 0.05 to 3 M, more preferably 0.1 to 1 M, and still more preferably 0.2 to 0.5 M.

### 1-2. Initiator

The sugar used as the initiator in the present invention encompasses a monosaccharide, a disaccharide, an oligosaccharide, and a polysaccharide.

The monosaccharide means a monosaccharide encompassing diose, and specific examples thereof include monosaccharides having 2 to 7 carbon atoms (that is, diose, triose, tetrose, pentose, hexose, and heptose). The monosaccharide may be either an aldose or a ketose. Furthermore, the monosaccharide may have a chain (linear or branched) structure or a cyclic structure.

Specific examples of the monosaccharide include glycolaldehyde, dihydroxyacetone, glyceraldehyde, erythrose, erythrulose, ribose, arabinose, xylose, lyxose, ribulose, xylulose, psicose, fructose, sorbose, tagatose, allose, altrose, glucose, mannose, gulose, idose, galactose, talose, and heptulose. These monosaccharides may be used singly or in combination of two or more kinds thereof. Among these monosaccharides, glycolaldehyde, dihydroxyacetone, fructose, sorbose, and glucose are preferred.

The disaccharide is one in which two monosaccharides are condensed. Specific examples of the disaccharide include lactose, maltose, isomaltose, sucrose, and trehalose. These disaccharides may be used singly or in combination of two or more kinds thereof. Among these disaccharides, sucrose is preferred.

The oligosaccharide is one in which 3 to 10 monosaccharides are condensed. Specific examples of the oligosaccharide include fructooligosaccharides, galactooligosaccharides, xylooligosaccharides, soybean oligosaccharides, isomaltooligosaccharides, lactosucrose, and raffinose. These oligosaccharides may be used singly or in combination of two or more kinds thereof.

The polysaccharide is one in which 11 or more monosaccharides are condensed. Specific examples of the polysaccharide include starch, dextrin, cellulose, glycogen, dextran, mutan, levan, and inulin. These polysaccharides may be used singly or in combination of two or more kinds thereof. Among these polysaccharides, starch is preferred.

In the present invention, as the sugar, any one of four kinds of sugars of monosaccharides, disaccharides, oligosaccharides, and polysaccharides may be used, or two or more kinds thereof may be used in combination. Among these sugars, from the viewpoint of improving the reaction efficiency of the formose reaction and/or the selectivity of the formose reaction, monosaccharides, disaccharides, and oligosaccharides are preferred, monosaccharides and disaccharides are more preferred, and monosaccharides are still more preferred.

Since it is considered that the production method of the present invention improves the reaction rate based on the promotion of the regeneration of the intermediate product (glycolaldehyde) by the promotion of the retro-aldol reaction (reaction represented by an arrow k4 in FIG. 1) in the formose reaction, the amount of the sugar to be contained in the reaction mixture may be about the amount of an initiator. The amount of the initiator used in the production method of the present invention is not particularly limited, but is, for example, 0.1 to 50 mmol, preferably 3 to 30 mmol, more preferably 5 to 15 mmol, and still more preferably 8 to 12 mmol, as an amount used per mol of charged formaldehyde.

### 1-3. Catalyst

The predetermined catalyst used in the production method of the present invention is a compound having an ability to deprotonate a hydroxyl group of erythrose and not having an ability to deprotonate water. By selecting a catalyst having such an appropriate base strength and using the catalyst together with a sugar as an initiator, it becomes possible to produce a sugar from formaldehyde under neutral conditions.

The specific base strength of the predetermined catalyst is pH 4 to 9, preferably pH 6.5 to 8.5 at 25° C. when the catalyst is prepared as an aqueous solution having a concentration of 60 mM.

Specific examples of the predetermined catalyst include a compound containing a metal selected from the group consisting of Group 5 elements, Group 6 elements, and Group 13 elements, and/or an organic base.

Specific examples of the compound containing the metal include an oxide of a Group 5 element, a Group 6 element, or a Group 13 element, an oxoacid of a Group 5 element, a Group 6 element, or a Group 13 element and/or a salt thereof, and/or a complex of a Group 5 element, a Group 6 element, or a Group 13 element.

Examples of the Group 5 elements include vanadium, niobium, tantalum, and dubnium. Examples of the Group 6 elements include chromium molybdenum, tungsten, and seaborgium. Examples of the Group 13 element include boron, aluminum, gallium, indium, thallium, and nihonium.

Examples of the salt of an oxoacid of a Group 5 element, a Group 6 element, or a Group 13 element include alkali metal salts such as potassium salts and sodium salts; and alkaline earth metal salts such as calcium and barium salts.

As for the complex of a Group 5 element, a Group 6 element, or a Group 13 element, the ligand is not particularly limited, and any of those known as polyoxometalates in which other metal atoms such as copper are incorporated or organometallic complexes is appropriately selected by those skilled in the art.

The compound containing the metal elements supply a metal ion in the formose reaction system constructed in the production method of the present invention. Thus, when the compound itself is a compound that is hardly soluble in water (for example, an alkaline earth metal salt), the solubility in water can be appropriately enhanced by using a chelating agent in combination. Such a chelating agent is not particularly limited, and examples thereof include ethylenediaminetetraacetic acid, nitrilotriacetic acid, hydroxyethylethylenediaminetriacetic acid, diethylenetriaminepentaacetic acid, crown ethers, and cryptands. These chelating agents may be used singly or in combination of two or more kinds thereof.

Specific examples of the organic base include organic amines. Specific examples of the organic amine include diethylaminoethanol, pyridine, and proline. These organic amines may be used singly or in combination of two or more kinds thereof.

Among the above-described catalysts, one catalyst may be used singly or a plurality of catalysts may be used in combination in the production method of the present invention.

Among the above catalysts, preferred are compounds containing the metal elements, more preferred are compounds containing metal elements selected from the group consisting of niobium, molybdenum, tungsten, and aluminum, still more preferred are niobium oxide, molybdenum oxide, tungsten oxide, aluminum oxide, niobic acid and salts thereof, molybdic acid and salts thereof, tungstic acid and salts thereof, aluminic acid and salts thereof, and further preferred are molybdenum oxide, tungsten oxide, aluminum oxide, niobate, molybdate, tungstic acid and salts thereof.

The form of the catalyst that acts on formaldehyde is not particularly limited. Examples thereof include a form of the compound itself (specifically, a form dissolved in water), a form in which the compound is supported on an insoluble carrier, and a form of an insoluble solid phase doped with an ion to be supplied to a reaction system by containing the compound as a constituent element of a material.

The material of the insoluble carrier is not particularly limited as long as it is insoluble in water, and examples thereof include inorganic compounds and resins, and more specific examples thereof include silica, alumina, and carbon materials. The material of the insoluble solid phase is not particularly limited, and examples thereof include apatite and talcite.

The amount of the catalyst used is not particularly limited, but is, for example, 50 to 400 mmol, preferably 100 to 300 mmol, more preferably 150 to 250 mmol, as an amount per mol of charged formaldehyde.

### 1-4. pH Conditions

In the production method of the present invention, the reaction mixture is subjected to neutral conditions to allow the formose reaction to proceed. The neutral conditions in the present invention mean that the pH of the reaction liquid at 25° C. is 6.5 to 8.5, preferably 7.3 to 8.0.

1-5. Other Conditions such as Reaction Conditions

Since it is considered that the production method of the present invention improves the reaction rate based on the promotion of the regeneration of the intermediate product (glycolaldehyde) by the promotion of the retro-aldol reaction in the formose reaction, it is not necessary to add a substrate other than formaldehyde to the reaction mixture. Thus, in a preferred embodiment of the production method of the present invention, a compound other than formaldehyde is not added as a substrate to the reaction mixture (specifically, it means that the intermediate substances shown in FIG. 1 are not added as a substrate, and this is distinguished from using a sugar as the initiator as described above).

Examples of the reaction solvent used in the reaction mixture include at least water, and from the viewpoint of suppressing side reactions, a mixed solution of water and a lower alcohol is preferred. Examples of the lower alcohol include methanol, ethanol, and isopropyl alcohol, and methanol is preferred. The amount of the lower alcohol contained in the mixed solution of water and the lower alcohol is, for example, 1 to 20 vol %, preferably 5 to 15 vol %, and more preferably 8 to 12 vol % from the viewpoint of suppressing side reactions.

The reaction temperature to which the reaction mixture is subjected is not particularly limited as long as the formose reaction can proceed, and the reaction temperature is, for example, 50 to 100° C., preferably 70 to 90° C., and more preferably 75 to 85° C. The reaction time may be appropriately determined according to the scale of the reaction mixture, the amount of produced sugar, and the like, and is, for example, 4 to 20 hours.

### 1-6. Other Steps

The production method of the present invention can be combined with an electrochemical reaction for producing formaldehyde by electrochemical reduction of carbon dioxide. That is, in this case, the production method of the present invention further includes the step of obtaining formaldehyde as the substrate of the above-described step by electrolysis of carbon dioxide (hereinafter, also described as “electrolysis reaction step”), and the electrolysis reaction step and the formose reaction step can be performed in the electrolysis reaction system.

Usually, while an aqueous solution of carbon dioxide is acidic to neutral, a conventional formose reaction using formaldehyde as the substrate proceeds under basic conditions. Thus, the formose reaction cannot proceed in the electrolysis reaction solution (that is, in the same reaction system). On the other hand, in the production method of the present invention, since the formose reaction step proceeds under neutral conditions, the electrolysis reaction step and the formose reaction step can be performed in the same reaction system, that is, in the electrolysis reaction system.

After completion of the reaction, the reaction mixture is cooled, and the produced sugar can be purified by a known method. Examples of the purification method include cation exchange chromatography, anion exchange chromatography, and silica gel column chromatography, and these are used singly or in combination of two or more kinds thereof depending on the components to be removed and the like.

### 1-7. Sugars Produced

The sugar produced in the method of the invention is typically a pentose and/or hexose.

### 2. Catalyst

As described above, the compound having an ability to deprotonate a hydroxyl group of erythrose and not having an ability to deprotonate water can allow the formose reaction to proceed further selectively in the production of a sugar under neutral conditions using formaldehyde as the substrate and using a sugar as the initiator. Therefore, the present invention also provides a catalyst which contains a compound having an ability to deprotonate a hydroxyl group of erythrose and not having an ability to deprotonate water and is used for production of a sugar under neutral conditions using formaldehyde as a substrate and using a sugar as an initiator.

Details of the compound having an ability to deprotonate a hydroxyl group of erythrose and not having an ability to deprotonate water, which is the entity (active ingredient) of the catalyst of the present invention, and details of the method for producing a sugar using the catalyst of the present invention are as described above in “1. Method for Producing Sugar”.

### 3. Apparatus for Producing Sugar

As described above, since the compound containing a metal selected from the group consisting of Group 5 elements and Group 6 elements can allow the formose reaction to proceed under neutral conditions, by combining the electrolysis reaction of carbon dioxide to formaldehyde with the above-described method for producing a sugar, the electrolysis reaction and the formose reaction can be performed in the same reaction system, that is, in the electrolysis reaction system. Therefore, the present invention also provides an apparatus for producing a sugar that contains a carbon dioxide electrolysis apparatus for obtaining formaldehyde from carbon dioxide, and a catalyst that is a compound containing a metal selected from the group consisting of Group 5 elements and Group 6 elements.

A carbon dioxide electrolysis apparatus for obtaining formaldehyde from carbon dioxide is well known to those skilled in the art, and any carbon dioxide electrolysis apparatus is appropriately selected by those skilled in the art. In addition, the catalyst is provided in any mode capable of contacting the catalyst with formaldehyde generated by the carbon dioxide decomposer. Details of the catalyst are as described above in “1. Method for Producing Sugar”. The apparatus for producing a sugar of the present invention is used for carrying out the production method according to an aspect in which an electrochemical reaction for producing formaldehyde by electrochemical reduction of carbon dioxide is combined among the aspects described above in “1. Method for Producing Sugar”.

### EXAMPLES

Hereinafter, the present invention will be described more specifically with reference to examples, but the present invention is not limited thereto.

Reagents and apparatuses used in the following test examples are as follows.

### Catalyst

Any of the following catalysts is a compound having an ability to deprotonate a hydroxyl group of erythrose and not having an ability to deprotonate water. In the following description, the numerical value shown in < > represents the pH at 25° C. of the catalyst that is prepared as an aqueous solution having a catalyst concentration of 60 mM.
Disodium tungstate dihydrate, <7.5> (Wako)
Disodium molybdate dihydrate, <7.5> (Wako)
Molybdenum oxide <7.5> (Wako)
Potassium niobate, <7.5> (8 potassium 6 niobium 10 oxide) (Mitsuwa Chemicals)
Aluminum oxide, <6.63> (sigma)
Barium tungstate, <7.5> (Mitsuwa Chemicals)
Tungstate anion immobilized carrier, <7.08>

The tungstate anion immobilized carrier was prepared as follows. A 5% aqueous NaOH solution, 10 mL, was added to 3.03 g of a strongly basic anion exchange resin, and the mixture was bubbled with argon for 7 minutes and then stirred for 50 minutes. After stirring, the aqueous NaOH solution was removed by decantation and the resin was stored in a refrigerator overnight. Next, the resin was washed 3 times with 10 mL of pure water. Subsequently, 7 mL of pure water was added, and a total of 0.67593 g of solid tungstic acid H₂WO₄ was added in several portions while stirring. The liquid was removed, and washing was conducted twice with 7 mL of pure water. After washing, ammonia water having pH 11.54 was added, and the mixture was stirred for 100 minutes. The ammonia water was removed, and washing was conducted three times with 7 mL of water. Pure water, 10 mL, was added to the resin, which was refrigerated overnight in an argon atmosphere. The liquid, 3 mL, was removed, and a total of 0.04421 g of tungstic acid was added in several portions while stirring. The liquid was removed, and washing was conducted six times with 7 mL of pure water.

### Initiator

Glycolaldehyde (trade name: Glycolaldehyde dimer) (sigma aldrich)
Dihydroxyacetone dimer (Tokyo Chemical Industry)
Fructose (Wako)
Sorbose (Wako)
Glucose (Wako)
Sucrose (Wako)
Starch (Wako)

### Substrate

Formaldehyde solution (Wako)

### Others

Disodium ethylenediaminetetraacetate (Wako)
2,4-Dinitrophenylhydrazine (Sigma-Aldrich)
Acetonitrile for high performance liquid chromatography (Wako)
Cation exchange resin Amberlite IR120B H AG (ORGANO)
Anion exchange resin Amberlite IRA402BL CI (ORGANO)
Anion exchange resin (strongly basic) (Wako)
Syringe filter (PTFE013022L AS ONE)
Organic synthetic stirrer (HHE-19G-USIV KPI)
High performance liquid chromatograph (Hitachi)

The formose reaction was carried out by the following operations.

In ultrapure water, 60 mM catalyst, a formaldehyde solution (such an amount as to give 0.3 M formaldehyde), and 3 mM initiator were dissolved to prepare 4 mL of an aqueous solution (reaction mixture (pH at 25° C. is about 7.5, neutral)). The reaction mixture was placed in a screw vial together with a stir bar and warmed with stirring at 80° C. using an organic synthesis stirrer (KPI). After completion of the reaction, the mixture was cooled with ice water, passed through a cation exchange resin (ORGANO) and an anion exchange resin (ORGANO), and further filtered through a syringe filter (AS ONE) to obtain a product mixture.

The product mixture was derivatized with DNPH as follows and prepared as a sample for analysis.

The product mixture, 2.5 μL, was dissolved in 748 μL of ultrapure water. To the solution, 375 μL of an acetonitrile solution of 2,4 dinitrophenylhydrazine (DNPH) (Sigma-Aldrich) (1 mg/1 mL) was added, and 20 μL of a 20% aqueous phosphoric acid solution was added. Thereafter, the mixture was stirred using a stirrer for 30 minutes to obtain a sample for analysis.

Formaldehyde and the formose reaction intermediate products up to tetrose were analyzed by subjecting the sample derivatized with DNPH as described above for analysis to HPLC. A high-performance liquid chromatograph (Hitachi) was used as the apparatus, and a UV/Vis detector (Hitachi) was used as the detector. Inertsustain C18 (GL Sciences) was used as the column. A mixed solution of acetonitrile for high-performance liquid chromatography (Wako) and ultrapure water in a ratio of 4:6 (volume ratio) was used as the mobile phase, and a flow rate was set to 1.0 mL/min.

The analysis of pentoses and hexoses was carried out by diluting the product mixture with the same amount of acetonitrile and subjecting it to HPLC. A high-performance liquid chromatograph Chromaster (registered trademark) sugar (phosphate-phenylhydrazine method) analysis system (Hitachi) was used as the apparatus. NH2P-50 4E (Shodex) was used as the column. As the mobile phase, acetonitrile for high-performance liquid chromatography (Wako), ultrapure water, and a 10% aqueous phosphoric acid solution were fed in a gradient, and the flow rate was set to 1.0 mL/min.

### Preliminary Test Example

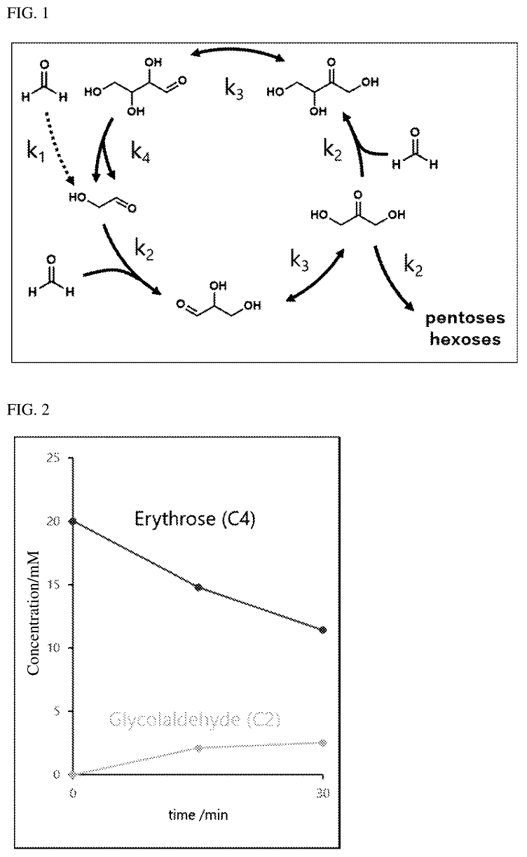

A reaction mixture was prepared by dissolving 20 mM erythrose and 60 mM disodium tungstate in water, and the reaction mixture was heated with an aluminum block at 80° C. to perform a retro-aldol reaction. The reaction mixture was derivatized with DNPH as described above and the temporal change in the concentration of each of erythrose (C4) and the product (glycolaldehyde (C2)) was examined. The results are shown in FIG. 2.

As shown in FIG. 2, production of glycolaldehyde (C2) was observed with reduction of erythrose (C4). That is, it has been revealed that the retro-aldol reaction proceeds by using disodium tungstate as the catalyst, and glycolaldehyde is obtained.

### Test Example 1

In a 10 vol % aqueous methanol solution, 0.3 M formaldehyde (HCHO) as the substrate, 3 mM glycolaldehyde (C2) as the initiator, and 60 mM disodium tungstate as the catalyst were dissolved to prepare a reaction mixture (pH at 25° C. was about 7.5), which was heated with an aluminum block at 80° C. to allow the formose reaction to proceed under neutral conditions.

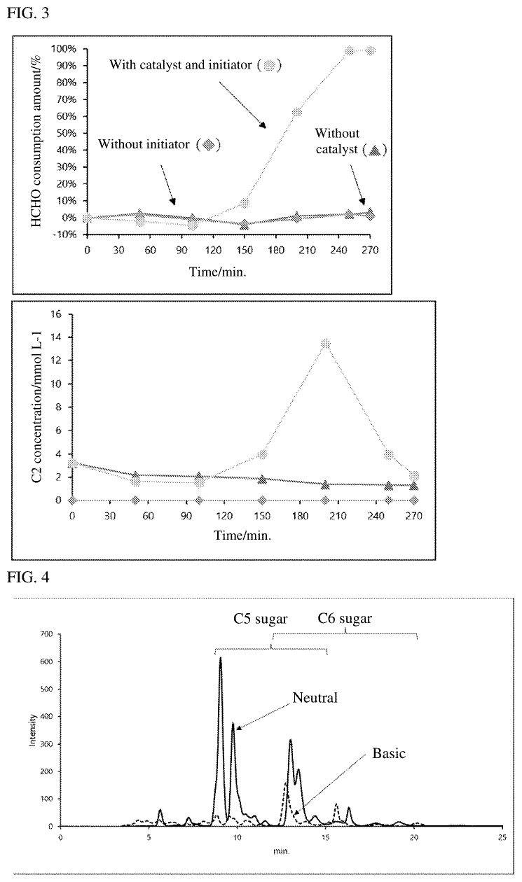

The amount of formaldehyde (HCHO) consumed and the concentration of glycolaldehyde (C2) produced as the formose reaction proceeded were examined over time by HPLC. The results are shown in FIG. 3. FIG. 3 also shows the results obtained in the case of carrying out the same procedure except that no initiator was used and the results obtained in the case of carrying out the same procedure except that no catalyst was used.

As shown in FIG. 3, it can be seen that formaldehyde was consumed over time and the formose reaction proceeded. The sigmoidal consumption curve of formaldehyde also suggests that the reaction proceeded autocatalytically. In addition, from the temporal change in the concentration of the glycolaldehyde, the production of glycolaldehyde in an amount exceeding the amount added as the initiator was confirmed with the progress of the reaction, and it was revealed that the regeneration of glycolaldehyde occurred due to formation of an autocatalytic cycle. On the other hand, clear progress of the reaction was not confirmed under either condition without a catalyst or condition without an initiator.

FIG. 4 shows results of HPLC analysis of pentoses and hexoses produced by the formose reaction at the time 99 mol % of formaldehyde was consumed (230 minutes). In FIG. 4, the results obtained in the case of carrying out the same procedure except that sodium hydroxide was used instead of disodium tungstate to provide basic conditions (pH 12.5 (25° C.)) (the results at the time 99 mol % of formaldehyde was consumed (17 minutes)) are also shown by a dotted line graph.

As shown in FIG. 4, it was found that by performing the formose reaction under neutral conditions using disodium tungstate and an initiator, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 2

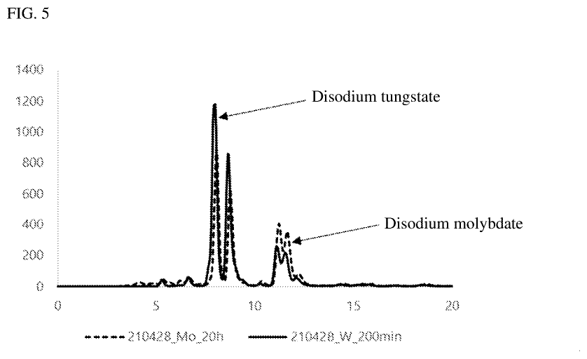

The same procedure as in Test Example 1 was carried out to perform the formose reaction under neutral conditions except that disodium molybdate was used as the catalyst instead of disodium tungstate, and pentoses and hexoses produced were analyzed by HPLC at the time 99 mol % of formaldehyde was consumed (20 hours). The results are shown in FIG. 5. In FIG. 5, the results of this Test Example are shown by a dashed line, and in addition, the results of Test Example 1 (the results of the formose reaction performed under neutral conditions using disodium tungstate) are also shown by a solid line.

As shown in FIG. 5, it was found that by performing the formose reaction under neutral conditions using disodium molybdate and an initiator, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 3

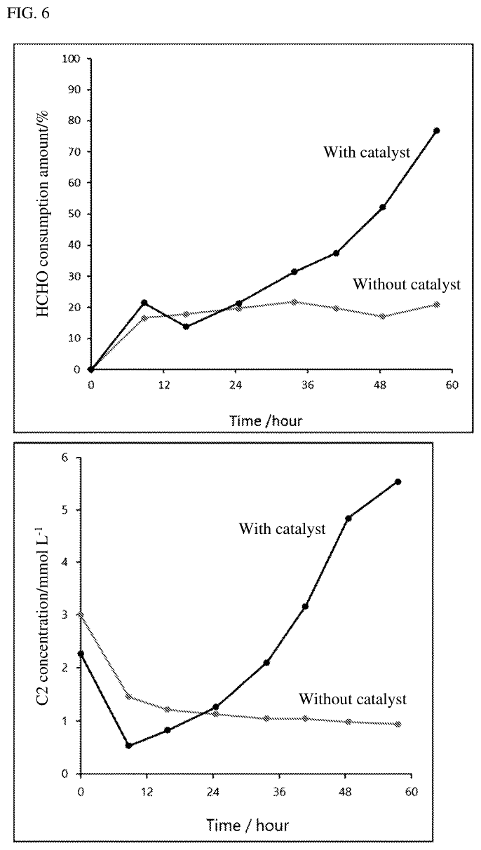

The same procedure as in Test Example 1 was carried out to perform the formose reaction under neutral conditions except that molybdenum oxide was used as the catalyst instead of disodium tungstate and the pH of the aqueous molybdenum oxide solution was adjusted to 7.5 with sodium hydroxide, and the amount of formaldehyde (HCHO) consumed and the concentration of glycolaldehyde (C2) as the formose reaction proceeded were examined over time by HPLC. The results are shown in FIG. 6. FIG. 6 also shows results when the same operation was performed except that no catalyst was used. As shown in FIG. 6, it can be seen that formaldehyde was consumed over time and the formose reaction proceeded. In addition, from the temporal change in the concentration of the glycolaldehyde, the production of glycolaldehyde in an amount exceeding the amount added as the initiator was confirmed with the progress of the reaction, and it was revealed that the regeneration of glycolaldehyde occurred due to the formation of an autocatalytic cycle. On the other hand, under conditions without a catalyst, neither clear progress of the reaction nor regeneration of glycolaldehyde was confirmed.

### Test Example 4

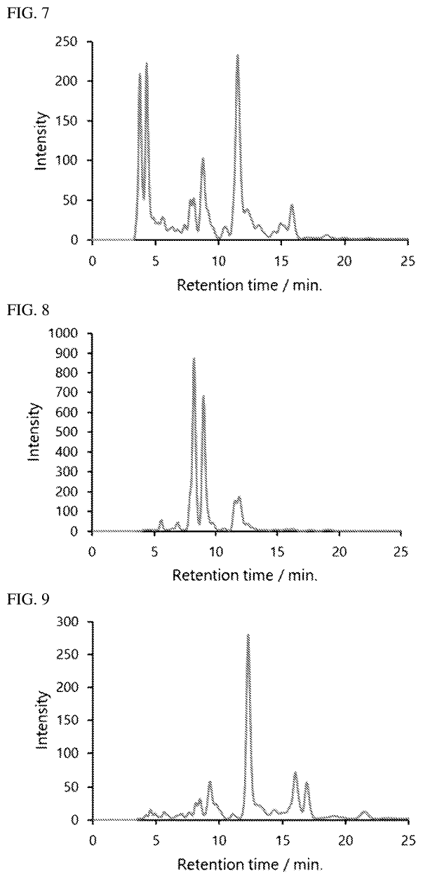

The same procedure as in Test Example 1 was carried out to perform the formose reaction under neutral conditions except that aluminum oxide was used as the catalyst instead of disodium tungstate, and the pH (25° C.) of the reaction mixture containing the catalyst, formaldehyde, and the initiator was adjusted to a neutral pH of 6.63, and pentoses and hexoses produced were analyzed by HPLC at the time 99 mol % of formaldehyde was consumed (72 hours). The results are shown in FIG. 7.

As shown in FIG. 7, it was found that by performing the formose reaction under neutral conditions using aluminum oxide and glycolaldehyde, the formose reaction selectively proceeded, and pentoses and hexoses were selectively produced.

### Test Example 5

The same procedure as in Test Example 1 was carried out to perform the formose reaction under neutral conditions except that barium tungstate solubilized with EDTA·2Na (100 mM) was used as the catalyst instead of disodium tungstate, and pentoses and hexoses produced were analyzed by HPLC at the time 99 mol % of formaldehyde was consumed (27 hours). The results are shown in FIG. 8.

As shown in FIG. 8, it was found that by performing the formose reaction under neutral conditions using barium tungstate and glycolaldehyde, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 6

The same procedure as in Test Example 1 was carried out to perform the formose reaction under neutral conditions except that potassium niobate was used as the catalyst instead of disodium tungstate, and pentoses and hexoses produced were analyzed by HPLC at the time 99 mol % of formaldehyde was consumed (68 hours). The results are shown in FIG. 9.

As shown in FIG. 9, it was found that by performing the formose reaction under neutral conditions using potassium niobate and glycolaldehyde, the formose reaction selectively proceeded, and pentoses and hexoses were selectively produced.

### Test Example 7

The same procedure as in Test Example 1 was carried out to perform the formose reaction under neutral conditions except that tungstate acid immobilized on an insoluble carrier was used as the catalyst instead of disodium tungstate, and the pH (25° C.) of the reaction mixture containing the catalyst, formaldehyde, and the initiator was adjusted to a neutral pH of 7.08, and pentoses and hexoses produced were analyzed by HPLC at the time 99 mol % formaldehyde was consumed (72 hours). The results are shown in FIG. 10.

As shown in FIG. 10, it was found that by performing the formose reaction under neutral conditions using tungstate acid and glycolaldehyde, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, and the like were contained.

### Test Example 8

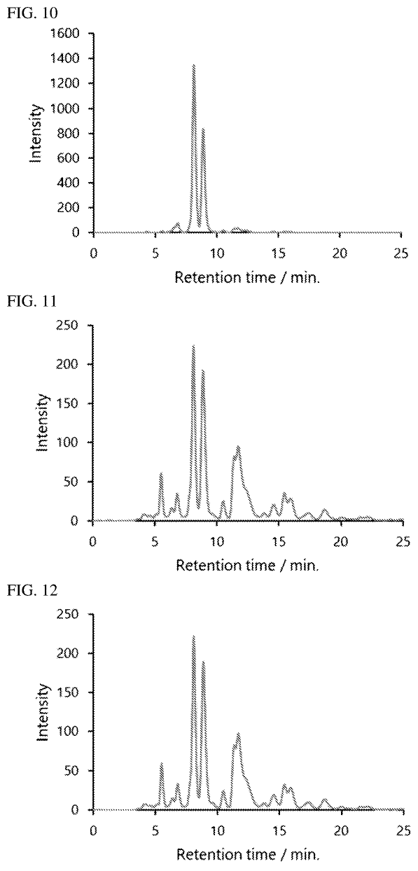

The same procedure as in Test Example 1 was carried out to perform the formose reaction under neutral conditions except that dihydroxyacetone was used as the initiator instead of glycolaldehyde, and pentoses and hexoses produced were analyzed by HPLC at the time 99 mol % formaldehyde was consumed (230 minutes). The results are shown in FIG. 11.

As shown in FIG. 11, it was found that by performing the formose reaction under neutral conditions using disodium tungstate and dihydroxyacetone, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 9

The same procedure as in Test Example 1 was carried out to perform the formose reaction under neutral conditions except that fructose was used as the initiator instead of glycolaldehyde, and pentoses and hexoses produced were analyzed by HPLC at the time 99 mol % of formaldehyde was consumed (230 minutes). The results are shown in FIG. 12.

As shown in FIG. 12, it was found that by performing the formose reaction under neutral conditions using disodium tungstate and fructose, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 10

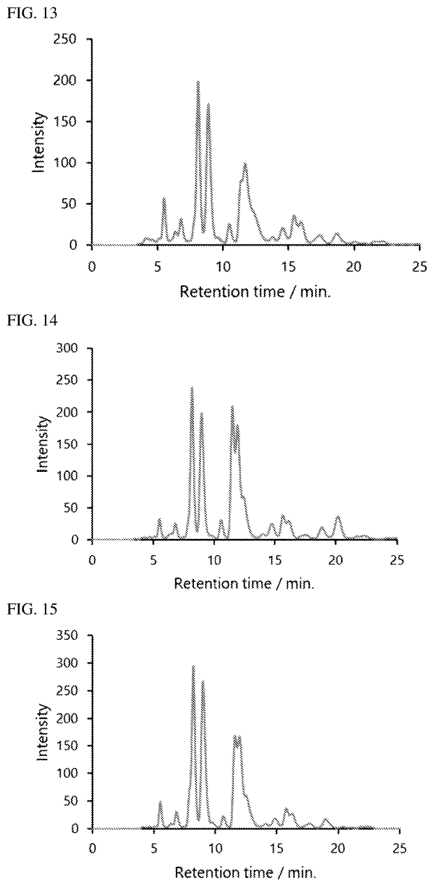

The same procedure as in Test Example 1 was carried out to perform the formose reaction under neutral conditions except that sorbose was used as the initiator instead of glycolaldehyde, and pentoses and hexoses produced were analyzed by HPLC at the time 99 mol % of formaldehyde was consumed (230 minutes). The results are shown in FIG. 13.

As shown in FIG. 13, it was found that by performing the formose reaction under neutral conditions using disodium tungstate and sorbose, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 11

The same procedure as in Test Example 1 was carried out to perform the formose reaction under neutral conditions except that glucose was used as the initiator instead of glycolaldehyde, and pentoses and hexoses produced were analyzed by HPLC at the time 99 mol % of formaldehyde was consumed (300 minutes). The results are shown in FIG. 14.

As shown in FIG. 14, it was found that by performing the formose reaction under neutral conditions using disodium tungstate and glucose, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 12

The same procedure as in Test Example 1 was carried out to perform the formose reaction under neutral conditions except that sucrose was used as the initiator instead of glycolaldehyde, and pentoses and hexoses produced were analyzed by HPLC at the time 99 mol % of formaldehyde was consumed (17 hours). The results are shown in FIG. 15.

As shown in FIG. 15, it was found that by performing the formose reaction under neutral conditions using disodium tungstate and sucrose, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

### Test Example 13

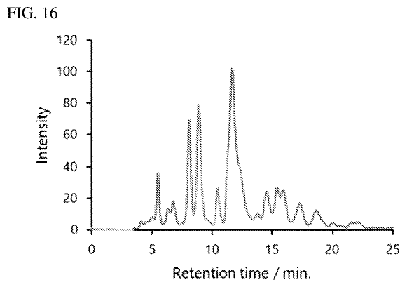

The same procedure as in Test Example 1 was carried out to perform the formose reaction under neutral conditions except that starch was used as the initiator instead of glycolaldehyde, and pentoses and hexoses produced were analyzed by HPLC at the time 99 mol % of formaldehyde was consumed (15 hours). The results are shown in FIG. 16.

As shown in FIG. 16, it was found that by performing the formose reaction under neutral conditions using disodium tungstate and starch, the formose reaction selectively proceeded, and pentoses and hexoses were selectively produced. The results of analysis of these pentoses and hexoses showed that 1,3,4-trihydroxy-3-(hydroxymethyl)butan-2-one, 1,3,4,5-tetrahydroxypentan-2-one, 1,2,4,5,6-pentahydroxyhexan-3-one, and the like were contained.

1. A method for producing a sugar, the method comprising a step of allowing a compound having an ability to deprotonate a hydroxyl group of erythrose and not having an ability to deprotonate water to act as a catalyst under neutral conditions, using formaldehyde as a substrate and a sugar as an initiator.

2. The method according to claim 1, wherein when the catalyst is prepared as an aqueous solution having a concentration of 60 mM, the catalyst has a base strength of pH 4 to 9 at 25° C.

3. The method according to claim 1, wherein the catalyst is a compound containing a metal selected from the group consisting of a Group 5 element, a Group 6 element, and a Group 13 element, and/or an organic base.

4. The method according to claim 3, wherein the compound containing the metal is an oxide of a Group 5 element, a Group 6 element, or a Group 13 element, an oxoacid of a Group 5 element, a Group 6 element, or a Group 13 element and/or a salt of the oxoacid, and/or a complex of a Group 5 element, a Group 6 element, or a Group 13 element.

5. The method according to claim 3, wherein the metal is selected from the group consisting of niobium, molybdenum, tungsten, and aluminum.

6. The method according to claim 3, wherein the compound containing the metal is selected from the group consisting of molybdate, tungstate, molybdenum oxide, niobate, and aluminum oxide.

7. The method according to claim 1, wherein the catalyst has a form that is a form of the compound itself, a form in which the compound is supported on an insoluble carrier, and/or a form of an insoluble solid phase doped with an ion to be supplied to a reaction system by containing the compound as a constituent element of a material.

8. The method according to claim 1, wherein the initiator is used in a ratio of from 0.1 to 50 mmol to 1 mol of the substrate.

9. The method according to claim 1, wherein a substrate other than formaldehyde is not added.

10. The method according to claim 1, further comprising a step of obtaining the formaldehyde by electrolysis of carbon dioxide, wherein the step of obtaining the formaldehyde and the step of allowing the compound to act as the catalyst are performed in a system of the electrolysis.

11. A catalyst that comprises a compound having an ability to deprotonate an hydroxyl group of erythrose and not having an ability to deprotonate water, and is used in production of a sugar under neutral conditions using formaldehyde as a substrate and a sugar as an initiator.

12. A sugar production system comprising: a carbon dioxide electrolysis apparatus that forms formaldehyde from carbon dioxide; and the catalyst according to claim 11.
